(19) United States
(12) Patent Application Publication
(10) Pub. No.: US 2026/0060578 A1
(43) Pub. Date: Mar. 5, 2026

(54) Momentary Stress Algorithm for a Wearable Computing Device

(71) Applicant: Google LLC, Mountain View, CA (US)

(72) Inventors: Samy Ahmed Mansour Abdel-Ghaffar, Berkeley, CA (US); Conor Joseph Heneghan, Campbell, CA (US); Lindsey Michelle Sunden, San Francisco, CA (US); David Duncanson Gutschick, Sunnyvale, CA (US); Qian He, Hayward, CA (US); Sarah Ann Stokes Kernasovskiy, San Francisco, CA (US); Seamus David Thomson, Mountain View, CA (US)

(21) Appl. No.: 19/104,477

(22) PCT Filed: Aug. 17, 2022
(86) PCT No.: PCT/US2022/040607

§ 371 Date: Feb. 18, 2025

(51) Int. Cl. A61B 5/16; A61B 5/00; A61B 5/0205; A61B 5/024; A61B 5/0531; G16H 40/67

(52) U.S. Cl. CPC A61B 5/165; A61B 5/02055; A61B 5/0531; A61B 5/681; A61B 5/7225; A61B 5/7264; A61B 5/742; G16H 40/67; A61B 5/02405

(57) ABSTRACT

A method of monitoring stress of a user includes receiving a plurality of time-series data inputs from a plurality of biometric sensor electrodes of a wearable computing device. The time-series data inputs includes continuous electrodermal activity data and at least one of heart rate data, skin temperature data, and heart rate variability data. The method also includes processing the time-series data inputs using a plurality of filtering techniques in sequence. Further, the method includes selecting a model from a plurality of models based on types of data inputs received as the time-series data inputs to calculate an indicator of a physiological response of the user at a certain time. Thus, the selected model is tailored to use all of the time-series data inputs in the calculation of the indicator of the physiological response. Further, the method includes controlling a function of the device when the indicator of the physiological response exceeds a threshold.

### FIELD

The present disclosure relates generally to wearable computing devices, and more particularly, to a momentary stress algorithm for a wearable computing device.

### BACKGROUND

Recent advances in technology, including those available through consumer devices, have provided for corresponding advances in health detection and monitoring. For example, biometric monitoring devices, such as fitness trackers and smart watches, are able to determine information relating to the pulse or motion of a person wearing the device.

Certain biometric monitoring devices include a variety of sensors for measuring multiple biological parameters that can be beneficial to a user of the device, such as a heart rate sensor, multi-purpose electrical sensors compatible with electrocardiogram (ECG) and electrodermal activity (EDA) applications, infrared sensors, a gyroscope, an altimeter, an accelerometer, a temperature sensor, an ambient light sensor, Wi-Fi, GPS, a vibration sensor, a speaker, and a microphone, among others. As one example, certain biometric monitoring devices measure EDA responses of the user's skin using a multi-path electrical sensor. These responses are observed as sensitive electrical changes to skin conductance, and are usually detected on the user's palm or fingertips using wet or dry electrode systems.

Typical EDA responses can be measured at the palm or fingertips using at least two electrodes, wherein skin conductance is calculated using the measured electrical impedance. EDA responses are represented as the phasic component of skin conductance—skin conductance responses (SCRs)—and are detected by identifying momentary spikes to skin conductance in comparison to a background tonic measurement, the skin conductance level (SCL). In general, SCRs are more accurately observed from data collected from a user's palm or fingertips due to high sweat gland density in these regions.

Although SCR detection at the palm or fingertips has been comprehensively reported in the literature for evaluating stress, the SCL alone can be beneficial for evaluating a user's stress. By measuring continuous electrodermal activity (cEDA), SCL can be used to observe certain biological events such as the body's response to acute stress events. However, using cEDA measurements for evaluating acute stress events is challenging if using electrodes that are mounted on a top face of a biometric monitoring device as cEDA needs continuous skin contact to provide accurate readings (i.e., requiring the user to have their skin positioned over the electrode surfaces for the duration of measurement). Electrodes for cEDA are thus positioned on the underside (skin-facing) side of a biometric monitoring device for the purposes of encouraging continuous contact with the skin, and not requiring frequent user input to facilitate continuous EDA measurement.

More particularly, in terms of timing, the primary difference between SCL and SCR is that SCRs occur on the scale of seconds, whereas SCL is evaluated across seconds, minutes, hours, and/or days. As an example, FIG. 1 illustrates a graphical representation of EDA amplitude versus time. As shown, the graph provides a comparison of the phasic skin conductance component (SCRs) represented as peaks to the tonic skin conductance component (SCL). Thus, accurately detecting changes to SCL needs to be continuously measured (over seconds/minutes/hours/days, etc.), and—with or without detection of SCRs—may be used for evaluation of physiological stress.

Accordingly, a wearable computing device that continuously measures EDA for the purpose of accurate detection of momentary or acute stress events and displays such events to a user would be welcomed in the art. Additionally, input of other sensors (e.g., photoplethysmography data (such as amplitude), accelerometer data, etc.) may provide additional context for evaluating acute stress events and/or their display to a user.

### SUMMARY

Aspects and advantages of embodiments of the present disclosure will be set forth in part in the following description, or can be learned from the description, or can be learned through practice of the embodiments.

In an aspect, the present disclosure is directed to a method of monitoring stress of a user using a wearable computing device. The method includes receiving, via a processor communicatively coupled to the wearable computing device, a plurality of time-series data inputs from a plurality of biometric sensor electrodes of the wearable computing device. The plurality of time-series data includes continuous electrodermal activity (cEDA) data of the user and at least one of heart rate data of the user, skin temperature data of the user, and heart rate variability (HRV) data of the user. The method also includes processing the plurality of time-series data inputs using a plurality of filtering techniques in sequence. Further, the method includes selecting a model from a plurality of models based on types of data inputs received as the plurality of time-series data inputs. Moreover, the method includes applying the selected model to the processed plurality of time-series data inputs to calculate an indicator of a physiological response of the user at a certain time, wherein the selected model is tailored to use all of the plurality of time-series data inputs in the calculation of the indicator of the physiological response. In addition, the method includes controlling a function of the wearable computing device when the indicator of the physiological response exceeds a threshold.

In another aspect, the present disclosure is directed to a wearable computing device. The wearable computing device includes an electronic display, a plurality of biometric sensor electrodes for sensing a plurality of time-series data inputs relating to biometrics of a user of the wearable computing device, and at least one processor communicatively coupled to the plurality of biometric sensor electrodes. The processor(s) is configured to perform a plurality of operations, including but not limited to receiving the plurality of time-series data inputs. The plurality of time-series data includes continuous electrodermal activity (cEDA) data of the user and at least one of heart rate data of the user, skin temperature data of the user, and heart rate variability (HRV) data of the user. The operations further include processing the plurality of time-series data inputs using a plurality of filtering techniques in sequence, selecting a model from a plurality of models based on types of data inputs received as the plurality of time-series data inputs, and applying the selected model to the processed plurality of time-series data inputs to calculate an indicator probability of a stress event of the user at a certain time by the user. Thus, the selected model is tailored to use all of the plurality of time-series data inputs in the calculation of the indicator probability of the stress event. In addition, the operations include controlling a function of the wearable computing device when the indicator of the stress event exceeds a threshold.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1 provides a graphical representation of electrodermal activity (EDA) amplitude (y-axis) versus time (x-axis) according to one embodiment of the present disclosure;

FIG. 2 provides a perspective view of a wearable computing device on a wrist of a user according to one embodiment of the present disclosure;

FIG. 3 provides a front perspective view of a wearable computing device according to one embodiment of the present disclosure;

FIG. 4 provides a rear perspective view of the wearable computing device of FIG. 3;

FIG. 5 provides an exploded view of the display of the wearable computing device of FIG. 3;

FIG. 6 provides a schematic diagram of an example set of devices that are able to communicate according to one embodiment of the present disclosure;

FIG. 7 illustrates various controller components of an example system that can be utilized according to one embodiment of the present disclosure;

FIG. 8 illustrates a flow diagram of an embodiment of a method of monitoring indicators of stress of a user using a wearable computing device according to the present disclosure.

FIG. 9 provides a flow chart of an embodiment of a momentary stress algorithm for calculating an indicator of stress of a user of a wearable computing device at a certain time according to the present disclosure.

FIG. 10 illustrates a graphical representation of an embodiment of unfiltered cEDA data of a user of a wearable computing device during an exercise event according to the present disclosure.

FIG. 11 illustrates a graphical representation of an embodiment of filtered cEDA of a user of a wearable computing device during an exercise event according to the present disclosure.

FIG. 12 provides a schematic diagram of normalization factors being transferred from a backend to a mobile device and to a momentary stress algorithm application of a wearable computing device according to the present disclosure.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

### Overview

Recent advances in technology, including those available through consumer devices, have provided for corresponding advances in health detection and monitoring. For example, devices such as fitness trackers and smart watches are able to determine information relating to the pulse or motion of a person wearing the device. Due to capabilities of conventional devices, however, the amount and types of health information able to be determined using such devices has been limited.

However, recent advances in sensor, electronics, and power source miniaturization have allowed the size of personal health monitoring devices to be offered in extremely small sizes that were previously impractical. For example, certain biometric monitoring devices include a wristband having a housing that is about 1.6″ wide by 1.6″ long by 0.5″ thick. Such biometric monitoring devices generally include a display, battery, sensors, electronics package, wireless communications capability, power source, and an interface button packaged within this small volume.

Moreover, certain biometric monitoring devices include a variety of sensors for measuring multiple biological parameters that can be beneficial to a user of the device, such as a heart rate sensor, multi-purpose electrical sensors compatible with ECG and EDA applications, infrared sensors, a gyroscope, an altimeter, an accelerometer, a temperature sensor, an ambient light sensor, Wi-Fi, GPS, a vibration sensor, a speaker, and a microphone, among others.

As one example, certain biometric monitoring devices measure EDA responses of the user's skin using a multi-path electrical sensor. These responses are observed as sensitive electrical changes to skin conductance, and are usually detected on the user's palm or fingertips using wet or dry electrode systems As such, EDA responses can be used to evaluate changes to physiological stress of a user.

Typical EDA responses can be measured at the palm or fingertips using at least two electrodes, wherein skin conductance is calculated using the measured electrical impedance. EDA responses are represented as the phasic component of skin conductance—skin conductance responses (SCRs)—and are detected by identifying momentary spikes to skin conductance in comparison to a background tonic measurement, the skin conductance level (SCL). In general, SCRs are more accurately observed from data collected from a user's palm or fingertips due to high sweat gland density in these regions.

Although SCR detection at the palm or fingertips has been comprehensively reported in the literature for evaluating stress, the SCL alone can be beneficial for evaluating a user's stress. By measuring continuous electrodermal activity (cEDA), SCL can be used to observe certain biological events such as the body's response to acute physiological responses, such as stress events. However, using cEDA measurements for evaluating acute stress events is challenging if using electrodes that are mounted on a top face of a biometric monitoring device as cEDA needs continuous skin contact to provide accurate readings (i.e. requiring the user to have their skin positioned over the electrode surfaces for the duration of measurement). Electrodes for cEDA are thus positioned on the underside (skin-facing) side of a biometric monitoring device for the purposes of encouraging continuous contact with the skin, and not requiring frequent user input to facilitate continuous EDA measurement.

Accordingly, the present disclosure is directed to a wearable computing device and a computer-implemented method for determining an indicator of stress (such as a probability of stress events exceeding a threshold, stress intensity, stress type, etc.) of a user at certain times. For example, the wearable computing device may implement a Momentary Stress Algorithm (MSA) programmed therein, either as an on-device algorithm or an in-mobile application, that is used to determine the indicator of a stress event. In particular, a MSA may be configured to predict physical (or physiological) signs of stress at a particular time and send a notification to the user of the predicted stress event. More specifically, in an embodiment, the MSA generally receives a combination of raw data inputs (e.g., heart rate data, cEDA data, skin temperature, acceleration, altimeter, and heart rate variability data) as time-series data that are processed using various filtering techniques. Filtering techniques may generally relate to eliminating (e.g., removing) unwanted data, e.g., data sets, from the raw data inputs and/or modifying (e.g., enhancing) certain data, e.g., part of the data and/or certain data sets, from the raw data inputs. Filtering the raw data inputs in sequence may accordingly include applying different filters one after the other, each applied filtering serves for eliminating and/or modifying values of the raw data inputs in a (algorithmically) pre-defined manner. For example, a filtering technique may comprise checking the raw data inputs against various modes of the device (such as sleep, exercise, do-not-disturb mode and/or off-wrist modes), such that data inputs collected during specific ones or all of these modes can be eliminated from consideration. The modes of the device to be taken into account for elimination may be selected by a user during use of the wearable computing device and/or be automatically associated to the raw input data by at least one processor of the wearable computing device (e.g., based on biometric sensor data).

For example, a user or the processor(s) may switch to a sleep mode, an exercise mode, a do-not-disturb mode or an off-wrist mode of the wearable computing device so that any input data collected during the respective mode is associated with this mode. Raw input data that was collected during a corresponding mode may then for example be generally considered not representative for indicating a physiological response, such as a stress event. In addition, the data inputs can be filtered using certain confounders so that data inputs corresponding to excessive movements (or a variety of other variables) can also be eliminated. In an example, if the MSA detects increased movement along with increased cEDA, the user is likely exercising (and not stressed) and such data can be excluded from the stress calculation.

With additional imputing utilized as needed, the final data set can also be normalized. In particular, in an embodiment, a mean and standard deviation (or any suitable normalization factors) may be required for each MSA data input at a time scale appropriate to that input. Moreover, for each input, various features can be calculated so as to transform the time series data set to a single value. Once the single value is obtained for each input, the MSA is configured to determine which model to use to estimate the indicator(s) of stress of the user. For example, the determination of the model to use can be based on what data inputs are available. Therefore, in an embodiment, if all of the input data is available (e.g., each of heart rate data, cEDA data, skin temperature, and heart rate variability data), the selected model is one in which all input data is used in the calculation of the indicator(s) of stress. In contrast, if only one or two input data sets are available, then the selected model is one in which only the two input data sets are used in the calculation of the indicator(s) of stress. The selected model (which may be a logistic regression classifier, as an example) can then be applied to the available data to determine the indicator(s) of stress of the user. In further embodiments, the MSA may also include post-processing or smoothing of the indicator(s) of stress of the user. Accordingly, the proposed solution comprises (automatically) selecting a model—for further evaluating the processed data—on the basis of the unprocessed data input, for example on the basis of a number of different input data types available or a number of time series of input data available. For example, in certain embodiments, the MSA may include requiring a detected stress event to be of a certain length (such as from about 3 minutes to about 5 minutes). In other embodiments, the MSA may group multiple stress events together in the event that the multiple stress events occur within a certain proximity to each other (e.g., the stress events are within 5 minutes of each other). Thus, in such embodiments, the MSA concludes that the multiple stress events represent a common stress event, rather than multiple back-to-back stress events.

Generally, a function of the wearable computing device may be controlled when the indicator of the stress event exceeds a threshold. As outlined above, such a function of the wearable computing device may be a function of a display of the wearable computing device, for example, resulting in that the indicator of the stress event is displayed at the display when the indicator of the stress event exceeds the threshold. Alternatively or additionally, the function of the wearable computing device controlled by the calculated indicator of the stress event exceeding the threshold may include generating and sending a stress event notification, e.g., to a user of the wearable computing device, and/or starting one or more (software) applications on the wearable computing device, e.g., for mood logging, for journaling, and/or for participation recording, and/or triggering a user interaction process via the wearable device in which the user of the wearable computing device has to actively confirm notification of the stress event. Thereby, a technique and a wearable computing device may be provided for making a user more efficiently aware of one or more potentially harmful stress events and automatically offering, in particular initiating countermeasures for decreasing a stress level of the user.

With reference now to the Figures, example embodiments of the present disclosure will be discussed in further detail.

### Example Devices and Systems

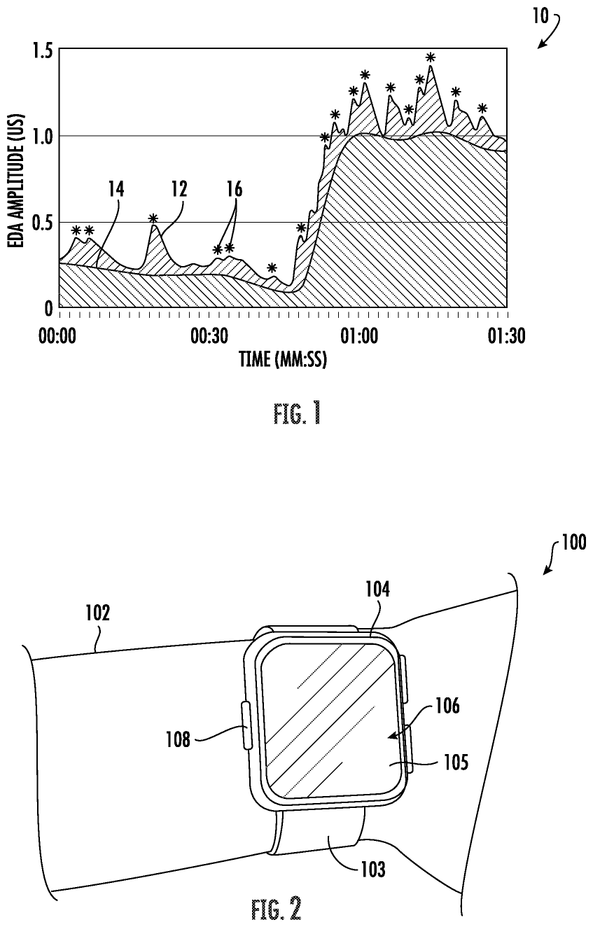

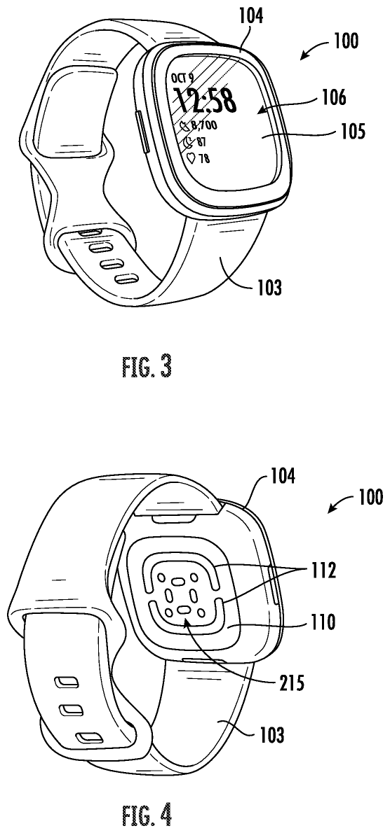

Referring now to the drawings, FIGS. 2-5 illustrate perspective views of a wearable computing device 100 according to the present disclosure. In particular, as shown in FIG. 2, the wearable computing device 100 may be worn on a user's forearm 102 like a wristwatch. Thus, as shown, the wearable computing device 100 may include a wristband 103 for securing the wearable computing device 100 to the user's forearm 102. In addition, as shown in FIGS. 2, 3, and 5, the wearable computing device 100 has an outer covering 105 and a housing 104 that contains the electronics associated with the wearable computing device 100. For example, in an embodiment, the outer covering 105 may be constructed of glass, polycarbonate, acrylic, or similar. Further, as shown in FIGS. 2, 3, and 5, the wearable computing device 100 includes an electronic display 106 arranged within the housing 104 and viewable through the outer covering 105. Moreover, as shown, the wearable computing device 100 may also include one or more buttons 108 that may be implemented to provide a mechanism to activate various sensors of the wearing computing device 100 to collect certain health data of the user. Moreover, in an embodiment, the electronic display 106 may cover an electronics package (not shown), which may also be housed within the housing 104.

Referring particularly to FIG. 4, the housing 104 of the wearable computing device 100 further includes a dorsal wrist-side face 110 configured to sit against a dorsal wrist of a user when being worn by the user and a plurality of sensor electrodes 112 positioned on the dorsal wrist-side face 110 of the housing 104 so as to maintain skin contact with the user when being worn on the wrist by the user. Thus, in such embodiments, each of the sensor electrodes 112 continuously measure, at least, electrical impedance of the user at a location of the skin contact on the dorsal wrist. Accordingly, in one or more embodiments, one or more (or all) of the plurality of sensor electrodes 112 may be cEDA sensor electrodes. In some embodiments, the wearable computing device 100 may also include at least one additional biometric sensor electrode in addition to the cEDA sensor electrodes. In such embodiments, the additional biometric sensor electrode may include one or more temperature sensors (such as an ambient temperature sensor or a skin temperature sensor), a humidity sensor, a light sensor, a pressure sensor, a microphone, an optical sensor, or a photoplethysmography (PPG) sensor.

Further, the sensor electrodes 112 described herein may be constructed of any suitable material. For example, in an embodiment, the sensor electrodes 112 described herein may be constructed of stainless steel, graphene, or any other material having a suitable conductivity and/or corrosion resistance and may have an optional PVD coating, that may be 1-micrometer thick titanium nitride. In such embodiments, the PVD coating may provide a desired color to the sensor electrodes 112, thereby preventing oxidation beyond what the stainless steel already provides, and also increases durability.

In additional embodiments, PVD and surface finish can be used to increase/decrease moisture retention, which affects the cEDA signal and user comfort. In particular embodiments, the sensor electrodes 112 may be formed of an alloy of tin and nickel (TiN) with a shiny or mirror surface finish. Moreover, in an embodiment, the sensor electrodes 112 may be constructed of a hydrophobic material or a transparent material.

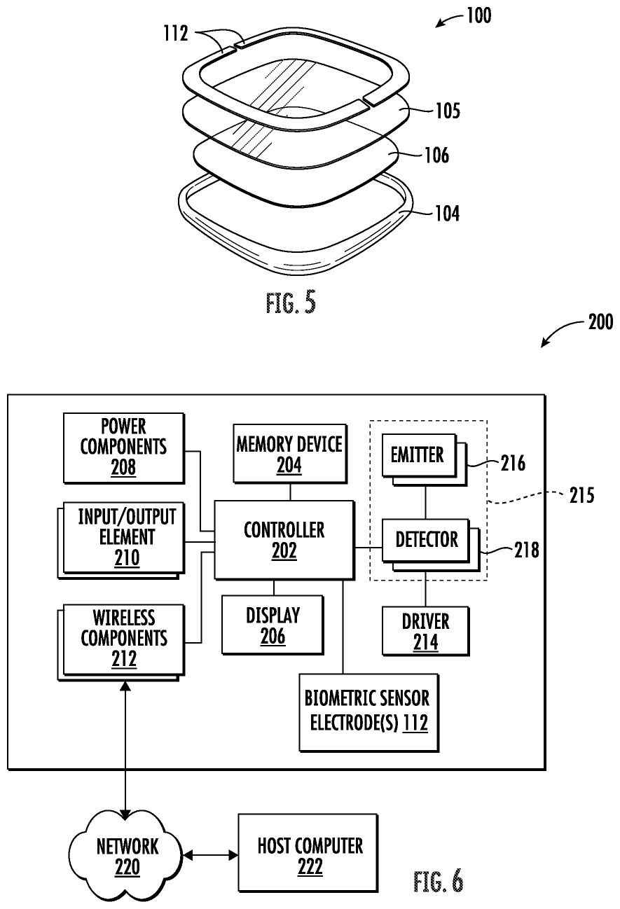

Referring now to FIG. 6, components of an example system 200 of the wearable computing device 100 that can be utilized in accordance with various embodiments are illustrated. In particular, as shown, the system 200 may also include at least one controller 202 communicatively coupled to the plurality of sensor electrodes 112. Moreover, in an embodiment, the controller(s) 202 may be a central processing unit (CPU) or graphics processing unit (GPU) for executing instructions that can be stored in a memory device 204, such as flash memory or DRAM, among other such options.

For example, in an embodiment, the memory device 204 may include RAM, ROM, FLASH memory, or other non-transitory digital data storage, and may include a control program comprising sequences of instructions which, when loaded from the memory device 204 and executed using the controller(s) 202, cause the controller(s) 202 to perform the functions that are described herein. As would be apparent to one of ordinary skill in the art, the system 200 can include many types of memory, data storage, or computer-readable media, such as data storage for program instructions for execution by the controller or any suitable processor. The same or separate storage can be used for images or data, a removable memory can be available for sharing information with other devices, and any number of communication approaches can be available for sharing with other devices.

In addition, as shown, the system 200 includes any suitable display 206, such as a touch screen, organic light emitting diode (OLED), or liquid crystal display (LCD), although devices might convey information via other means, such as through audio speakers, projectors, or casting the display or streaming data to another device, such as a mobile phone, wherein an application on the mobile phone displays the data.

The system 200 may also include one or more wireless components 212 operable to communicate with one or more electronic devices within a communication range of the particular wireless channel. The wireless channel can be any appropriate channel used to enable devices to communicate wirelessly, such as Bluetooth, cellular, NFC, Ultra-Wideband (UWB), or Wi-Fi channels. It should be understood that the system 200 can have one or more conventional wired communications connections as known in the art.

The system 200 also includes one or more power components 208, such as may include a battery operable to be recharged through conventional plug-in approaches, or through other approaches such as capacitive charging through proximity with a power mat or other such device. In further embodiments, the system 200 can also include at least one additional I/O device 210 able to receive conventional input from a user. This conventional input can include, for example, a push button, touch pad, touch screen, wheel, joystick, keyboard, mouse, keypad, or any other such device or element whereby a user can input a command to the system 200. In another embodiment, the I/O device(s) 210 may be connected by a wireless infrared or Bluetooth or other link as well in some embodiments. In some embodiments, the system 200 may also include a microphone or other audio capture element that accepts voice or other audio commands. For example, in particular embodiments, the system 200 may not include any buttons at all, but might be controlled only through a combination of visual and audio commands, such that a user can control the wearable computing device 100 without having to be in contact therewith. In certain embodiments, the I/O elements 210 may also include one or more of the sensor electrodes 112 described herein, optical sensors, barometric sensors (e.g., altimeter, etc.), and the like.

Still referring to FIG. 6, the system 200 may also include a driver 214 and at least some combination of one or more emitters 216 and one or more detectors 218 (referred to herein as an optics package 215) for measuring data for one or more metrics of a human body, such as for a person wearing the wearable computing device 100. In such embodiments, as shown in FIG. 4, for example, the optics package 215 may be arranged within the housing 104 and at least partially exposed through the dorsal wrist-side face 110 of the housing 104. Thus, as shown and further explained herein, the sensor electrodes 112 may be positioned around the optics package 215 on the wrist-side face 110 of the housing 104. In alternative embodiments, the various components of the optics package 215 may be positioned around the sensor electrodes 112 and/or in another other suitable configuration such as adjacent to, interspersed with, surrounded by, or on top of the optics package 215. In certain embodiments, for example, wherein the sensor electrodes 112 are transparent, the sensor electrodes 112 may be arranged atop the optics package 215.

In some embodiments, the system 200 may include at least one imaging element, such as one or more cameras that are able to capture images of the surrounding environment and that are able to image a user, people, or objects in the vicinity of the device. The imaging element can include any appropriate technology, such as a CCD image capture element having a sufficient resolution, focal range, and viewable area to capture an image of the user when the user is operating the device. Further image capture elements may also include depth sensors. Methods for capturing images using a camera element with a computing device are well known in the art and will not be discussed herein in detail. It should be understood that image capture can be performed using a single image, multiple images, periodic imaging, continuous image capturing, image streaming, etc. Further, the system 200 can include the ability to start and/or stop image capture, such as when receiving a command from a user, application, or other device.

The emitters 216 and detectors 218 of FIG. 6 may also be capable of being used, in one example, for obtaining optical PPG measurements. Some PPG technologies rely on detecting light at a single spatial location, adding signals taken from two or more spatial locations, or an algorithmic combination thereof. Both of these approaches result in a single spatial measurement from which the heart rate (HR) estimate (or other physiological metrics) can be determined. In some embodiments, a PPG device employs a single light source coupled to a single detector (i.e., a single light path). Alternatively, a PPG device may employ multiple light sources coupled to a single detector or multiple detectors (i.e., two or more light paths). In other embodiments, a PPG device employs multiple detectors coupled to a single light source or multiple light sources (i.e., two or more light paths). In some cases, the light source(s) may be configured to emit one or more of green, red, infrared (IR) light, as well as any other suitable wavelengths in the spectrum (such as long IR for metabolic monitoring). For example, a PPG device may employ a single light source and two or more light detectors each configured to detect a specific wavelength or wavelength range. In some cases, each detector is configured to detect a different wavelength or wavelength range from one another. In other cases, two or more detectors are configured to detect the same wavelength or wavelength range. In yet another case, one or more detectors configured to detect a specific wavelength or wavelength range different from one or more other detectors). In embodiments employing multiple light paths, the PPG device may determine an average of the signals resulting from the multiple light paths before determining an HR estimate or other physiological metrics.

Moreover, in an embodiment, the emitters 216 and detectors 218 may be coupled to the controller 202 directly or indirectly using driver circuitry by which the controller 202 may drive the emitters 216 and obtain signals from the detectors 218. The host computer 222 can communicate with the wireless networking components 212 via the one or more networks 220, which may include one or more local area networks, wide area networks, UWB, and/or internetworks using any of terrestrial or satellite links. In some embodiments, the host computer 222 executes control programs and/or application programs that are configured to perform some of the functions described herein.

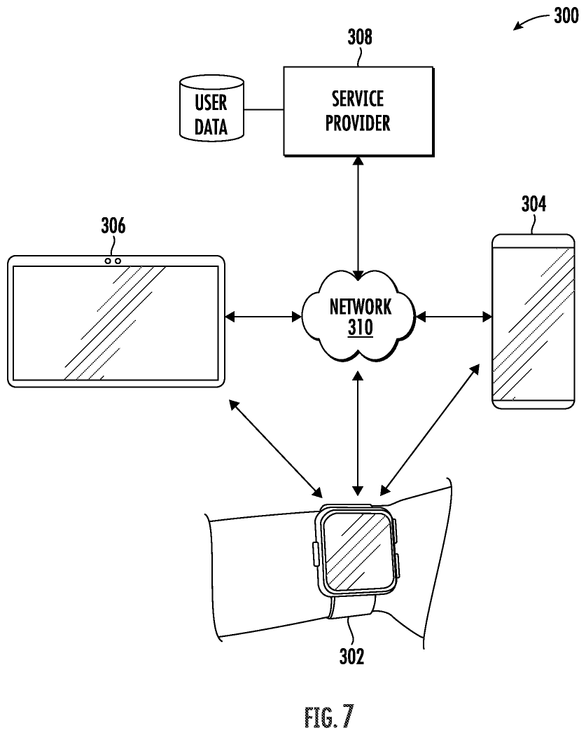

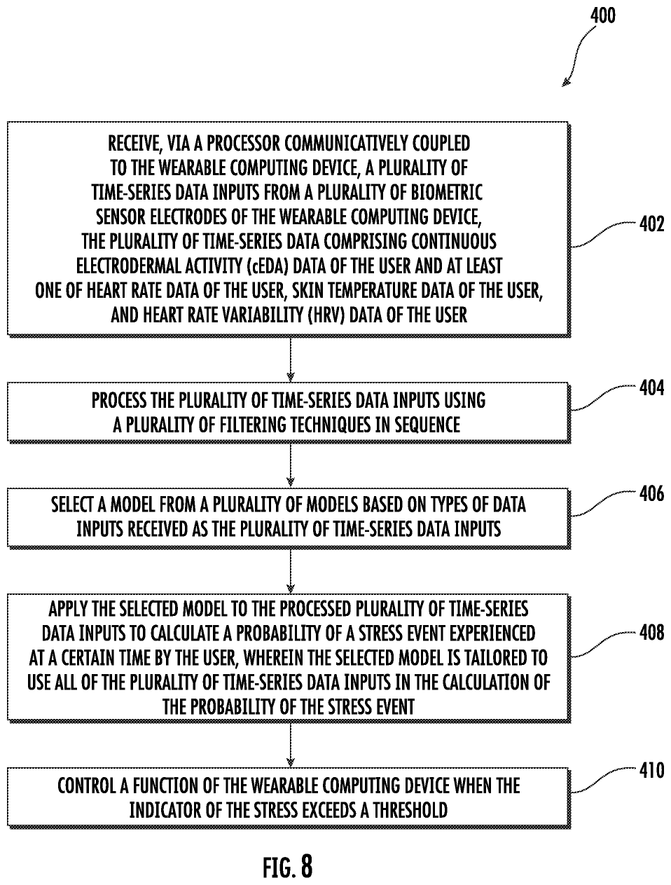

Referring now to FIG. 7, a schematic diagram of an environment 300 in which aspects of various embodiments can be implemented is illustrated. In particular, as shown, a user might have a number of different devices that are able to communicate using at least one wireless communication protocol. For example, as shown, the user might have a smartwatch 302 or fitness tracker (such as wearable computing device 100), which the user would like to be able to communicate with a smartphone 304 and a tablet computer 306. The ability to communicate with multiple devices can enable a user to obtain information from the smartwatch 302, e.g., data captured using a sensor on the smartwatch 302, using an application installed on either the smartphone 304 or the tablet computer 306. The user may also want the smartwatch 302 to be able to communicate with a service provider 308, or other such entity, that is able to obtain and process data from the smartwatch and provide functionality that may not otherwise be available on the smartwatch or the applications installed on the individual devices. In addition, as shown, the smartwatch 302 may be able to communicate with the service provider 308 through at least one network 220, such as the Internet or a cellular network, or may communicate over a wireless connection such as Bluetooth® to one of the individual devices, which can then communicate over the at least one network. There may be a number of other types of, or reasons for, communications in various embodiments.

In addition to being able to communicate, a user may also want the devices to be able to communicate in a number of ways or with certain aspects. For example, the user may want communications between the devices to be secure, particularly where the data may include personal health data or other such communications. The device or application providers may also be required to secure this information in at least some situations. The user may want the devices to be able to communicate with each other concurrently, rather than sequentially. This may be particularly true where pairing may be required, as the user may prefer that each device be paired at most once, such that no manual pairing is required. The user may also desire the communications to be as standards-based as possible, not only so that little manual intervention is required on the part of the user but also so that the devices can communicate with as many other types of devices as possible, which is often not the case for various proprietary formats. A user may thus desire to be able to walk in a room with one device and have such device automatically communicate with another target device with little to no effort on the part of the user. In various conventional approaches, a device will utilize a communication technology such as Wi-Fi to communicate with other devices using wireless local area networking (WLAN). Smaller or lower capacity devices, such as many Internet of Things (IoT) devices, instead utilize a communication technology such as Bluetooth®, and in particular Bluetooth Low Energy (BLE) which has very low power consumption.

In further embodiments, the environment 300 illustrated in FIG. 7 enables data to be captured, processed, and displayed in a number of different ways. For example, data may be captured using sensors on the smartwatch 302, but due to limited resources on the smartwatch 302, the data may be transferred to the smartphone 304 or the service provider 308 (or a cloud resource) for processing, and results of that processing may then be presented back to that user on the smartwatch 302, smartphone 304, and/or another such device associated with that user, such as the tablet computer 306. In at least some embodiments, a user may also be able to provide input such as health data using an interface on any of these devices, which can then be considered when making that determination.

Referring now to FIG. 8, a flow diagram of one embodiment of a method 400 of monitoring stress of a user using a wearable computing device is provided. In an embodiment, for example, the wearable computing device may be any suitable wearable computing device, such as the wearable computing device 100 described herein with reference to FIGS. 1-7. Thus, in general, the method 400 is described herein with reference to the wearable computing device 100 of FIGS. 1-7. However, it should be appreciated that the disclosed method 400 may be implemented with any other suitable wearable computing device having any other suitable configurations. In addition, although FIG. 8 depicts steps performed in a particular order for purposes of illustration and discussion, the methods discussed herein are not limited to any particular order or arrangement. One skilled in the art, using the disclosures provided herein, will appreciate that various steps of the methods disclosed herein can be omitted, rearranged, combined, added, and/or adapted in various ways without deviating from the scope of the present disclosure.

As mentioned, and described herein, the wearable computing device includes a plurality of biometric sensor electrodes on a dorsal wrist-side face of a housing of the wearable computing device. Thus, as shown at (402), the method 400 includes receiving, via a processor communicatively coupled to the wearable computing device 100, a plurality of time-series data inputs from a plurality of biometric sensor electrodes of the wearable computing device 100. In such embodiments, as an example, the plurality of time-series data may include cEDA data of the user as well as heart rate data of the user, skin temperature data of the user, heart rate variability (HRV) data of the user, accelerometer data, altimeter data, and/or combinations thereof. In particular embodiments, for example, the plurality of time-series data inputs may include the heart rate of the user, the skin temperature of the user, the heart rate variability of the user, and the cEDA data of the user, with the combination of such data inputs providing an improved estimation of a user's stress.

As shown at (404), the method 400 includes processing the plurality of time-series data inputs using a plurality of filtering techniques in sequence. For example, as will be described in more detail herein, processing the plurality of time-series data inputs using the plurality of filtering techniques in sequence may include filtering the cEDA data of the user, e.g., using a high-pass filter for SCR, a low-pass filter for SCL, a median filter to erase glitches, and/or any other type of filter as needed.

Further, in an embodiment, processing the plurality of time-series data inputs using the plurality of filtering techniques in sequence may include updating a certain time frame cache with the plurality of data inputs (i.e., updating a cache storing data inputs relating to time frame/time window of predetermined length), determining whether a time-series data input from the plurality of time-series data inputs indicate one of a plurality of modes of the wearable computing device relating to undesirable motion (e.g., from exercise) and, if so, eliminating the time-series data input from the plurality of time-series data inputs, filtering the plurality of time-series data inputs based on a plurality of confounders and eliminating or modifying time-series data inputs of the plurality of time-series data inputs that satisfy one or more of the plurality of confounders, imputing one or more data points into the plurality of time-series data inputs if a minimum number of data points are missing from the plurality of time-series data inputs or dropping the plurality of time-series data inputs if a certain number of data points are missing from the plurality of time-series data inputs, normalizing the plurality of time-series data inputs using one or more normalization factors, the one or more normalization factors comprising at least one of a mean, a median, a mode, or a standard deviation, and/or transforming each of the plurality of time-series data inputs into a single value.

Still referring to FIG. 8, as shown at (406), the method 400 includes selecting a model from a plurality of models based on types or values of data inputs received as the plurality of time-series data inputs. As shown at (408), the method 400 includes applying the selected model to the processed plurality of time-series data inputs to calculate an indicator of a physiological response, such as a stress event, at a certain time by the user, wherein the selected model is tailored to use all of the plurality of time-series data inputs in the calculation of the indicator of the stress event.

As shown at (410), the method 400 includes providing the indicator of the stress event at the certain time to the user via a display. More specifically, in an embodiment, the method 400 may include sending a notification to the user indicating at least one of the indicator of the stress event, a graphical representation of stress events over time, and/or a summary of stress events over time (such as daily, weekly, monthly, etc.). Moreover, in an embodiment, the method 400 may also include probing the user to respond to the notification via the display (such as display 206). For example, the user may be prompted to mood log, journal, record participation in a prescribed stress-relieving activity (such as meditating, walking, listening to music, guided breathing, etc.), and/or any other suitable response.

Generally, a function of the wearable computing device may be controlled when the indicator of the stress event exceeds a threshold. As outlined above, such a function of the wearable computing device may be a function of display 206 of wearable computing device 100, for example, resulting in that the indicator of the stress event is displayed at display 206 when the indicator of the stress event exceeds the threshold. Alternatively or additionally, the function of a wearable computing device 100 controlled by the calculated indicator of the stress event exceeding the threshold may include generating and sending a stress event notification, e.g., to a user of the wearable computing device, and/or starting one or more (software) applications on the wearable computing device 100, e.g., for mood logging, for journaling, and/or for participation recording, and/or triggering a user interaction process via the wearable computing device 100 in which the user of the wearable computing device 100 has to actively confirm notification of the stress event. Thereby, a technique and a wearable computing device 100 may be provided for making a user more efficiently aware of one or more potentially harmful stress events and automatically offering or even automatically initiating countermeasures for decreasing a stress level of the user.

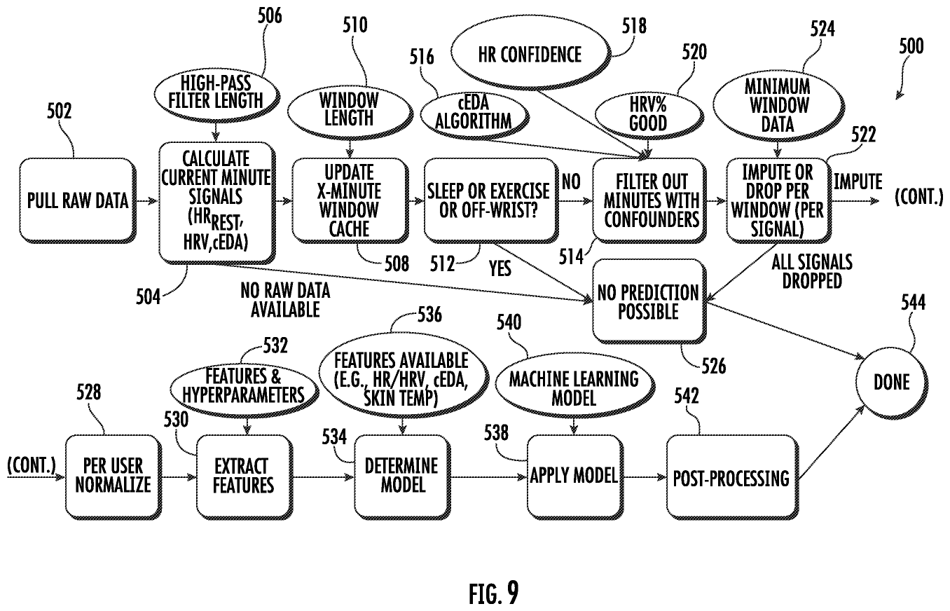

The method 400 of FIG. 8 can be better understood with respect to FIGS. 9-11. More specifically, FIG. 9 illustrates a flow chart of an embodiment of a momentary stress algorithm 500 for calculating an indicator of a stress event of a user of a wearable computing device at a certain time according to the present disclosure. In particular, as shown at (502), the algorithm 500 includes ingesting on-device data from the wearable computing device 100. As mentioned, such raw data may include, for example, cEDA data of the user as well as heart rate data of the user, skin temperature data of the user, accelerometer data, altimeter data, and/or HRV data of the user, and combinations thereof.

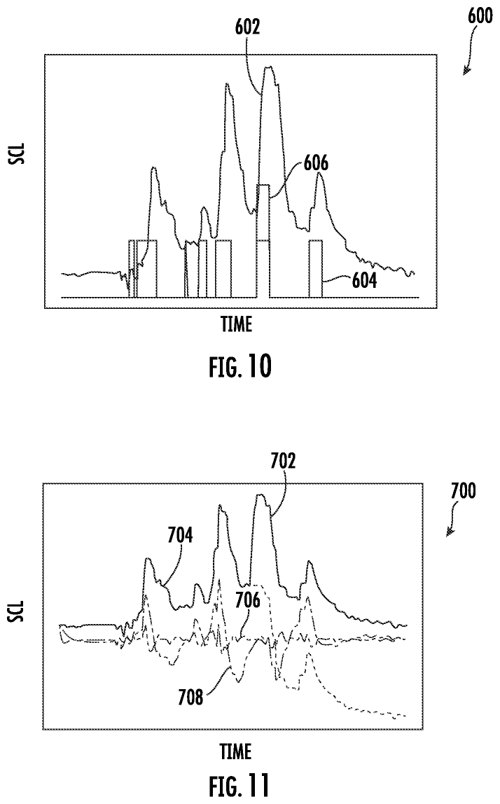

As shown at (504), the algorithm 500 includes calculating current minute signals for certain of the raw data (such as the heart rate data, HRV data, and/or the cEDA data). More specifically, in an embodiment, as shown at (506), the algorithm 500 may include applying a filter to the cEDA data, e.g., using a high-pass filter for SCR, a low-pass filter for SCL, a median filter to erase glitches, and/or any other type of filter as needed. For example, as shown in FIGS. 10 and 11, graphical representations 600, 700 of an embodiment of cEDA data (e.g., SCL; y-axis) of a user of the wearable computing device versus time (x-axis) during an exercise event according to the present disclosure are illustrated, respectively. However, FIG. 10 illustrates the raw data, whereas FIG. 11 illustrates filtered data, e.g., via a high-pass filter. Further, as shown in FIG. 10, SCL data is represented by 602, cEDA event data, such as a stress event, is represented by 604, and exercise detection is represented by 606. In FIG. 11, SCL data is represented by 702, cEDA/SCL data is represented by 704, and the high pass filtered data is represented by 706 and 708, respectively. In particular, as shown, the difference between the lines 706 and 708 is the cutoff frequency used to generate 706 versus 708, where 708 has a lower cutoff frequency (e.g., 120 minutes), meaning that changes over a longer time interval are maintained. Said differently, line 706 returns to zero about six times faster than line 708.

Accordingly, as shown in FIGS. 10 and 11, the cEDA/SCL data signal 602 can decay very slowly (e.g., hours). Therefore, the algorithm 500 may filter out exercise events from the cEDA data, without losing all predictive power of the “down-slope” side of the peak, which may include useful data. However, if linear interpolation between the start of the exercise and the “down-slope” side of the peak is used, the algorithm 500 will essentially re-introduce the exercise data. Thus, to address this issue, the high-pass filter may be applied to the cEDA signal 602, after e.g., the data has passed through a slew rate limiter or similar, to avoid introducing a sharp change in value or slope before filtering that data because such a change would be preserved by the filtering, leading to the appearance of a sharp spike in post-filtering values. In a simplistic sense, the algorithm monitors for a change in state such that sharp spikes would cause many erroneous predictions of stress events. The output, represented by 706 and 708, is the filtered cEDA data.

If no raw data is available (or enough raw data is not available), the algorithm 500 ends at (526) as no prediction is possible. However, if the raw data is available, the algorithm 500 continues at (508). In particular, as shown at (508), the algorithm 500 updates a certain time window (e.g., X-minute window) cache for one or more of the data inputs. Furthermore, in an embodiment, as shown at (510), the algorithm 500 may receive a window length (such as 30 minutes) that is set to update the cache. It should be understood that the window length can be selected as any suitable time frame and is not limited to 30 minutes.

Further, as shown at (512), the algorithm 500 includes determining whether a time-series data input from the raw data indicates one of a plurality of modes of the wearable computing device 100 relating to motion. In such embodiments, for example, the modes of the wearable computing device 100 may include a sleep mode, an exercise mode, or an off-wrist mode. If yes, the algorithm 500 ends at (526) as no prediction is possible. However, if no, the algorithm 500 continues at (514).

In particular, as shown at (514), if the wearable computing device 100 is not operating in one of the aforementioned modes, the algorithm 500 is configured to filter the raw data to remove certain minutes of the raw data based on a plurality of confounders and eliminate or modify the raw data that satisfies one or more of the plurality of confounders. In such embodiments, for example, the plurality of confounders may include the cEDA data of the user increasing with increased motion as shown at (516) (as generally shown by the graphical representation 600 of FIG. 10), a percentage of the HRV data being above a certain threshold as shown at (520), a certain confidence of the heart rate data of the user as shown at (518), the wearable computing device 100 being partially or fully submerged in liquid detectable using a combination of cEDA and altimeter data, skin contact between the wearable computing device 100 and/or the user based on how much of each minute the sensor electrodes 112 simultaneously have any contact with the user's skin, or humidity being above a humidity threshold.

Referring still to FIG. 9, as shown at (522), the algorithm 500 is further configured to impute one or more data points into the raw data if a certain number of data points are missing from the raw data or drop the raw data if the number of missing data points exceeds a threshold. Thus, as shown, if the number of missing data points exceeds the threshold the algorithm 500 ends at (526) as no prediction is possible. However, if the raw data is missing few enough values/data points, the algorithm 500 imputes all missing data points, e.g., using interpolation or extrapolation, and continues at (528).

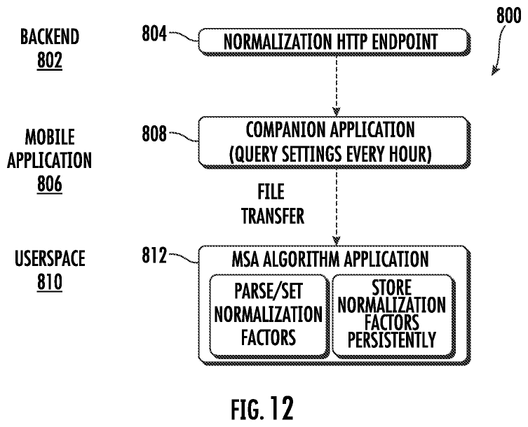

In particular, as shown at (528), the algorithm 500 is further configured to normalize the raw data using one or more normalization factors. In such embodiments, for example, the normalization factor(s) may include a mean, a median, a mode, a standard deviation, or any other suitable statistical function over a period of time suitable for each input. For example, as shown in FIG. 12, the normalization factors can be transported via file transfer, similar to how the settings can be transferred to the wearable computing device 100. In the illustrated embodiment, for example, a transportation mechanism 800 requires a backend 802 to expose a HTTP endpoint 804 that can be queried by a companion application 808, e.g., on a mobile device 806. The companion application 806 can then query these values at predefined time intervals and any change in payload results in the companion application 808 sending the normalization factors to the wearable computing device 100. The wearable computing device 100 can decode the file, persistently store the new normalization weights, and immediately apply them to the algorithm 500, e.g., via an MSA algorithm application 812 in a userspace 810.

Accordingly, referring back to FIG. 9, as shown at (530), the algorithm 500 is configured to extract certain features from the raw data by applying a number of commonly used time-series transformation functions. In particular, as shown at (532), the algorithm 500 is configured to receive (or may be programmed with) certain features and/or hyperparameters that can be used to extract the certain features from the raw data. In an embodiment, for example, the algorithm 500 is configured to transform each of the time-series raw data inputs into a single value. Thus, the algorithm 500 is configured to apply certain time-series transformation functions to each of the input signals independently.

Thus, as shown at (534), the algorithm 500 is then configured to determine/select a model from a plurality of models based on the types and/or values of data inputs received in the raw data. For example, as shown at (536), if the raw data includes heart rate data, HRV data, cEDA data, and skin temperature, the selected model is configured to use all of the received raw data to determine the indicator of a stress event experienced at a certain time by the user. In other embodiments, if less raw data is available (such as just cEDA data and skin temperature), then the algorithm 500 is configured to select a model that uses a subset of the available data types (such as the cEDA data and the skin temperature) to determine the indicator of a stress event experienced at a certain time by the user. Thus, it should be understood that the algorithm is capable of selecting the model that is tailored to the raw data available.

Thus, as shown at (538), the algorithm 500 is configured to apply the selected model to the data to generate an output, which may represent at physical (or physiological) sign of stress of a user. In further embodiments, as an example, the selected model may be a machine learning model. For example, as shown at (540), the machine learning model may be a logistic regression model, a deep neural network, or any other suitable machine learning model now known or later developed in the art.

As shown at (542), the algorithm 500 is also configured to post-process the indicator of the stress event of the user. In such embodiments, for example, post-processing the indicator of the stress event of the user may include ensuring that the stress event has a duration above a certain threshold. In another embodiment, post-processing the indicator of the stress event of the user may include grouping multiple stress events together if the multiple stress events occur within a certain time frame of each other. The algorithm 500 ends at (544).

Accordingly, and as mentioned, the output of the algorithm is an indicator of a stress event experienced by the user at a certain time. Thus, in further embodiments, the wearable computing device 100 can send a notification to the user indicating the indicator of the stress event, a graphical representation of stress events over time, and/or a summary of stress events over time. Moreover, as mentioned, the wearable computing device 100 can probe the user to respond to the notification via the display, such as by requesting the user to participate in mood logging, journaling, and recording participation in a prescribed stress-relieving activity.

### Additional Disclosure

Further to the descriptions above, a user may be provided with controls allowing the user to make an election as to both if and when systems, programs, or features described herein may enable collection of user information (e.g., information about a user's social network, social actions, or activities, profession, a user's preferences, or a user's current location), and if the user is sent content or communications from a server. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over what information is collected about the user, how that information is used, and what information is provided to the user. To that end, any information collected as described herein relating to the user will be kept private and confidential and will not be improperly used or published.

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure covers such alterations, variations, and equivalents.

1. A method of monitoring stress of a user using a wearable computing device, the method comprising:
receiving, via a processor communicatively coupled to the wearable computing device, a plurality of time-series data inputs from a plurality of biometric sensor electrodes of the wearable computing device, the plurality of time-series data comprising continuous electrodermal activity (cEDA) data of the user and at least one of heart rate data of the user, skin temperature data of the user, and heart rate variability (HRV) data of the user;
processing the plurality of time-series data inputs using a plurality of filtering techniques in sequence;
selecting a model from a plurality of models based on types of data inputs received as the plurality of time-series data inputs;
applying the selected model to the processed plurality of time-series data inputs to calculate an indicator of a physiological response of the user at a certain time, wherein the selected model is tailored to use all of the plurality of time-series data inputs in the calculation of the indicator of the physiological response; and
controlling a function of the wearable computing device when the indicator of the physiological response exceeds a threshold.

2. The method of claim 1, wherein the plurality of time-series data inputs comprise the heart rate of the user, the skin temperature of the user, the heart rate variability of the user, and the cEDA data of the user.

3. The method of claim 1, wherein processing the plurality of time-series data inputs using the plurality of filtering techniques in sequence further comprise:
filtering the cEDA data of the user using a high-pass filter, a low-pass filter, or a median filter.

4. The method of claim 1, wherein processing the plurality of time-series data inputs using the plurality of filtering techniques in sequence further comprise:
updating a certain time frame cache with the plurality of data inputs.

5. The method of claim 1, wherein processing the plurality of time-series data inputs using the plurality of filtering techniques in sequence further comprise:
determining whether a time-series data input from the plurality of time-series data inputs indicate one of a plurality of modes of the wearable computing device and, if so, eliminating or modifying the time-series data input from the plurality of time-series data inputs.

6. The method of claim 5, wherein the plurality of modes of the wearable computing device comprise one of a sleep mode, an exercise mode, a do-not-disturb mode, or an off-wrist mode.

7. The method of claim 1, wherein processing the plurality of time-series data inputs using the plurality of filtering techniques in sequence further comprise:
filtering the plurality of time-series data inputs based on a plurality of confounders and eliminating or modifying time-series data inputs of the plurality of time-series data inputs that satisfy one or more of the plurality of confounders.

8. The method of claim 7, wherein the plurality of confounders comprise one of the cEDA data of the user increasing with increased motion, a percentage of the HRV data being above a certain threshold, a certain confidence of the heart rate data of the user, a motion classifier based on accelerometer values, the wearable computing device being partially or fully submerged in liquid or exposed to the liquid, skin contact between the wearable computing device and the user being below a contact threshold, or humidity being above a humidity threshold.

9. The method of claim 1, wherein processing the plurality of time-series data inputs using the plurality of filtering techniques further comprise:
imputing one or more data points into the plurality of time-series data inputs if a certain number of data points are missing from the plurality of time-series data inputs or dropping the plurality of time-series data inputs if the number of missing data points exceeds a threshold.

10. The method of claim 1, wherein processing the plurality of time-series data inputs using the plurality of filtering techniques further comprise:
normalizing the plurality of time-series data inputs using one or more normalization factors.

11. The method of claim 1, wherein the selected model is a machine learning model.

12. The method of claim 1, wherein processing the plurality of time-series data inputs using the plurality of filtering techniques further comprise:
transforming each of the plurality of time-series data inputs into a single value.

13. The method of claim 1, further comprising post-processing the indicator of the physiological response, wherein post-processing the indicator of the physiological response further comprises at least one ensuring that the physiological response comprises a duration above a certain threshold and grouping multiple physiological responses together if the multiple physiological responses occur within a certain time frame of each other.

14. The method of claim 1, wherein controlling the function of the wearable computing device comprises at least one of controlling a display of the wearable computing device and providing the indicator of the physiological response at the certain time to the user via the display.

15. The method of claim 14, further comprising sending a notification to the user via the display indicating at least one of an occurrence of the indicator of the physiological response exceeding a threshold, a graphical representation of physiological responses over time, and a summary of physiological responses over time.

16. The method of claim 14, further comprising prompting the user to respond to the notification via the display of the wearable computing device, wherein a response to the notification comprises at least one of mood logging, journaling, guided breathing, guided meditation, and recording participation in a prescribed stress-relieving activity.

17. The method of claim 1, wherein the processor is part of one of the wearable computing device or a separate mobile device.

18. A wearable computing device, comprising:
an electronic display;
a plurality of biometric sensor electrodes for sensing a plurality of time-series data inputs relating to biometrics of a user of the wearable computing device; and
at least one processor communicatively coupled to the plurality of biometric sensor electrodes, the at least one processor configured to perform a plurality of operations, the plurality of operations comprising:
receiving the plurality of time-series data inputs, the plurality of time-series data comprising continuous electrodermal activity (cEDA) data of the user and at least one of heart rate data of the user, skin temperature data of the user, and heart rate variability (HRV) data of the user;
processing the plurality of time-series data inputs using a plurality of filtering techniques in sequence;
selecting a model from a plurality of models based on types of data inputs received as the plurality of time-series data inputs;
applying the selected model to the processed plurality of time-series data inputs to calculate an indicator probability of a stress event of the user at a certain time by the user, wherein the selected model is tailored to use all of the plurality of time-series data inputs in the calculation of the indicator probability of the stress event; and
controlling a function of the wearable computing device when the indicator of the stress event exceeds a threshold.

19. The wearable computing device of claim 18, wherein processing the plurality of time-series data inputs using the plurality of filtering techniques in sequence further comprise:
filtering the cEDA data of the user using a high-pass filter, a low-pass filter, or a median filter;
updating a certain time frame cache with the plurality of data inputs;
determining whether a time-series data input from the plurality of time-series data inputs indicate one of a plurality of modes of the wearable computing device and, if so, eliminating or modifying the time-series data input from the plurality of time-series data inputs;
filtering the plurality of time-series data inputs based on a plurality of confounders and eliminating or modifying time-series data inputs of the plurality of time-series data inputs that satisfy one or more of the plurality of confounders;
imputing one or more data points into the plurality of time-series data inputs if a certain number of data points are missing from the plurality of time-series data inputs or dropping the plurality of time-series data inputs if the number of missing data points exceeds a threshold;
normalizing the plurality of time-series data inputs using one or more normalization factors, the one or more normalization factors comprising at least one of a mean, a median, a mode, or a standard deviation for a time scale suitable to each time-series data input; and
transforming each of the plurality of time-series data inputs into a single value.

20. The wearable computing device of claim 18, or further comprising post-processing the indicator probability of the stress event, wherein post-processing the indicator probability of the stress event further comprises at least one ensuring that the stress event comprises a duration above a certain threshold and grouping multiple stress events together if the multiple stress events occur within a certain time frame of each other.

21-22. (canceled)
